(19) United States
(12) Patent Application Publication
(10) Pub. No.: US 2010/0298674 A1
(43) Pub. Date: Nov. 25, 2010

(54) PROTECTIVE SHELL FOR AN IN VIVO SENSOR MADE FROM RESORBABLE POLYMER

(75) Inventors: Arthur E. Colvin, JR., Mt. Airy, MD (US); Jason D. Colvin, Vienna, VA (US)

Correspondence Address: ROTHWELL, FIGG, ERNST & MANBECK, P.C., 1425 K STREET, N.W., SUITE 800, WASHINGTON, DC, 20005, US

(73) Assignee: Sensors for Medicine & Science, Inc., Germantown, MD (US)

(21) Appl. No.: 12/764,389

(22) Filed: Apr. 21, 2010

Related U.S. Application Data
(60) Provisional application No. 61/171,143, filed on Apr. 21, 2009.

(51) Int. Cl. A61B 5/145; A61B 5/1459

(52) U.S. Cl. 600/316; 600/365

(57) ABSTRACT

An implantable device with in vivo functionality, where the functionality of the device is negatively affected by the inflammation reaction generally associated with tissue injury, encapsulated by a protective coating that prevents damage to the device from any inflammation reactions. The protective coating is designed to persist for a set period of time, generally until after the inflammation reaction of the surrounding in vivo environment in response to the injury caused by the implantation procedure has concluded. The protective coating is further designed to “resorb” (i.e. to dissociate from the device, dissolve, and be absorbed into the surrounding environment) after a set period of time, allowing the device to perform its in vivo functionality unhindered without loss of performance.

This application claims the benefit of prior-filed provisional patent application U.S. 61/171,143 which was filed on Apr. 21, 2009, the contents of which are hereby incorporated by reference.

### BACKGROUND OF THE INVENTION

1. Field of the Invention

The present invention relates to devices designed to be implanted into the body of an animal. More particularly, the invention relates to (but is not in all cases necessarily limited to) electro-optical-based sensing devices for detecting the presence or concentration of an analyte in a medium which are characterized by being totally self-contained, with a smooth and rounded, oblong, oval, or elliptical shape (e.g., a bean- or pharmaceutical capsule-shape) and an extraordinarily compact size which permit the device to be implanted in humans for in situ detection of various analytes.

2. Description of Related Art

None of the references described or referred to herein are admitted to be prior art to the claimed invention.

Implantable devices for monitoring various physiological conditions are known. They include, for example, the sensors described in U.S. Pat. No. 5,517,313 to Colvin; U.S. Pat. No. 5,910,661 to Colvin; U.S. Pat. No. 5,917,605 to Colvin; U.S. Pat. No. 5,894,351 to Colvin; U.S. Pat. No. 6,304,766 to Colvin; U.S. Pat. No. 6,344,360 to Colvin et al.; U.S. Pat. No. 6,330,464 to Colvin; U.S. Pat. No. 6,400,974 to Lesho; U.S. Pat. No. 6,794,195 to Colvin; U.S. Pat. No. 7,135,342 to Colvin et al.; U.S. Pat. No. 6,940,590 to Colvin et al.; U.S. Pat. No. 6,800,451 to Daniloff et al.; U.S. Pat. No. 7,375,347 to Colvin et al.; U.S. Pat. No. 7,157,723 to Colvin et al.; U.S. Pat. No. 7,308,292 to Colvin et al.; and U.S. Pat. No. 7,190,445 to Colvin et al.; and in the following U.S. patent application Ser. No. 10/332,619 to Lesho filed Jun. 27, 2001; Ser. No. 10/824,587 to Colvin et al. filed Apr. 15, 2004; Ser. No. 10/822,670 to Colvin et al. filed Apr. 13, 2004; Ser. No. 10/825,648 to Colvin et al. filed Apr. 16, 2004; Ser. No. 10/923,698 to Colvin et al. filed Aug. 24, 2004; Ser. No. 11/447,980 to Waters et al. filed Jun. 7, 2006; Ser. No. 11/487,435 to Merical et al. filed Jul. 17, 2006; Ser. No. 12/043,289 to J. Colvin et al. filed Mar. 6, 2008; Ser. No. 11/948,419 to Colvin et al. filed Nov. 30, 2007; Ser. No. 11/925,272 to Colvin filed Oct. 26, 2007; and Ser. No. 61/084,100 to Colvin filed Jul. 28, 2008; the contents of all of the foregoing are incorporated by reference herein.

When a foreign object enters a body, there is an immediate immunological (i.e., inflammation) response to eliminate that foreign object. When the foreign object is an intentionally implanted device or sensor, the inflammation response can cause damage to or otherwise negatively impact the functionality of the implant. Thus, a need exists for an implantable device that can endure the biochemical activity of an inflammation response, such that the efficacy and useful life of the device is not adversely impacted by the inflammation response. A corresponding need exists for a method of manufacturing or treating an implantable device such that it can endure the biochemical activity of an inflammation response without significant loss of efficacy or useful life.

### SUMMARY OF THE INVENTION

Aspects of the invention are embodied, but not limited to, the various forms of the invention described below.

In one aspect, the present invention relates to a device comprising:
(a) an implantable device which has an in vivo functionality and;
(b) a layer of protective coating applied onto the implantable device wherein:
   (1) the protective coating prevents or reduces degradation or interference of the implantable device from inflammation reactions; and
   (2) the protective coating is designed to resorb over a period of time under in vivo conditions.

In another aspect, the present invention relates to a method for using an implantable device in in vivo applications comprising:
(a) providing an implantable device which has an in vivo functionality, and which comprises a layer of a protective coating on the device wherein:
   (1) the protective coating prevents or reduces degradation or interference of the device from inflammation reactions; and
   (2) during use the protective coating resorbs into the surrounding environment over a period of time; and
(b) implanting the implantable device in a subject body.

In another aspect, the present invention relates to a method for detecting the presence or concentration of an analyte in an in vivo sample, said method comprising:
a) exposing the sample to a device having a detectable quality that changes when the device is exposed to the analyte, said device comprising a layer of protective coating applied onto the implantable device wherein:
   (1) the protective coating prevents or reduces degradation or interference of the device from inflammation reactions; and
   (2) the protective coating is designed to resorb over a period of time under in vivo conditions,
such that the device has enhanced resistance to degradation or interference as compared to a corresponding device without the protective coating; and
b) measuring any change in said detectable quality to thereby determine the presence or concentration of said analyte in said sample.

These and other features, aspects, and advantages of the present invention will become apparent to those skilled in the art after considering the following detailed description, appended claims and accompanying drawings.

### BRIEF DESCRIPTION OF THE DRAWINGS

FIG. 1 is a graph showing loss of signal over time from a glucose sensor which is not treated according to an embodiment of the invention, immediately following implantation in a living body.

FIG. 2 is a schematic representation of the inflammation reaction of a living body in response to tissue injury, and indicates how molecules associated with the inflammation reaction may affect an implanted sensor.

FIG. 3 is an illustration of the chemical reaction where an unprotected —B(OH)₂ recognition element of a glucose indicator is oxidized when exposed to in vivo reactive oxygen radical species (ROS).

FIG. 4 is a picture of several sensors according to an embodiment of the invention with a polymer indicator layer grafted onto the clear substrate of the sensor, wherein the polymer layer causes the indicator light to scatter, appear white, and become brighter.

FIG. 5 is an electron micrograph of the graft membrane structure that is applied to the indicator and causes the bright, scattered, white light as seen in FIG. 4.

FIG. 6 is a pair of pictures showing, side-by-side, two sensors according to an embodiment of the invention, where the sensor on the left has been implanted in a human and subsequently removed, and where the sensor on the right is a control example that has never been implanted.

FIG. 7 is a pair of pictures, showing a single sensor in a before-after sequence, where the “before” picture is of the sensor after it has been implanted in a human and subsequently removed, and where the “after” picture is of the same sensor after it has been implanted, removed, and treated with a protease, wherein the protease has removed proteins that bound to the sensor while implanted.

FIG. 8A is a scanning electron microscope (SEMS) image of the polymer layer grafted onto a sensor according to an embodiment of the invention where the sensor has never been implanted, i.e., is a control image of the graft layer.

FIG. 8B is a SEMS image of the polymer layer grafted onto a sensor according to an embodiment of the invention where the sensor has been implanted in a human and the layer is opaque.

FIG. 8C is a SEMS image of the polymer layer grafted onto a sensor according to an embodiment of the invention where the sensor has been implanted in a human and the layer is clear due to infilling of the graft pores.

FIGS. 9A & 9B are a pair of SEMS images both showing a layer of protective coating (about 5 microns thick) sprayed onto the surface of an indicator graft membrane according to an embodiment of the invention.

FIG. 10A is an illustration of a chemical reaction for the synthesis of sebacic acid prepolymer, a polyanhydride.

FIG. 10B is an illustration of a chemical reaction for the synthesis of poly(sebacic acid), a polyanhydride.

FIG. 10C is an illustration of a chemical reaction for the synthesis of 1,3-bis(p-carboxyphenoxy)propane prepolymer.

FIG. 10D is an illustration of a chemical reaction for the synthesis of poly(1,3-bis(p-carboxyphenoxy)propane-co-sebacic acid), a polyanhydride.

FIG. 11 is a schematic representation of a semi-automated custom built extrusion set up comprising a heating block, extrusion plate and up-down slide jig used to thermally apply polymers onto a device according to embodiments of the invention.

FIGS. 12A and 12B contain illustrations of examples of preferred indicator molecules for use in the present invention.

### DETAILED DESCRIPTION OF THE INVENTION

The present invention is embodied in apparatus, and methods of using such apparatus, designed to be implanted into a living body and to perform an in vivo functionality. Such a system is described in the present disclosure in the context of an implantable sensor, and more specifically in the context of an implantable glucose monitoring sensor. While the in vivo functionality of the device described herein is that of a glucose detection sensor, embodiments of the present invention are not limited to only implantable glucose sensors and are not even limited to only implantable sensors.

The purpose of this invention is to protect an implantable sensor or device which may be either destroyed, weakened (either signal or mechanical strength), or suffer diminished function or utility as a result of normal body inflammation reaction stimulated by the implantation procedure.

Devices useful in the practice of the present invention include those described in the patents and publications listed above (para. [0006]) and incorporated by reference herein. In a preferred embodiment, the device is an implantable glucose monitoring sensor such as the sensor described in U.S. Pat. No. 6,330,464. The components that comprise the sensor include a sensor body, a matrix layer coated over the exterior surface of the sensor body, fluorescent indicator molecules distributed throughout the matrix layer, a radiation source, and a photosensitive detector element that generates signal indicative of the level of fluorescence in the indicator molecules. The indicator molecules may either be on the surface of the matrix layer, or contained within the matrix layer. The sensor body may be formed from a suitable, optically transmissive polymer material with a refractive index sufficiently different from that of the medium in which the sensor will be used, such that the polymer will act as an optical wave guide. In a preferred embodiment, the sensor may also have a power source to power the radiation source as well as a transmitter that can transmit a signal to an external receiver based on the photosensitive detector. The sensor body may completely encapsulate the radiation source and photosensitive detector as well as the power source and transmitter if present, crating a self-contained device.

For embodiments of the invention where the device is a sensor as described in U.S. Pat. No. 6,330,464, the specific composition of the matrix layer and the indicator molecules may vary depending on the particular analyte the sensor is to be used to detect and/or where the sensor is to be used to detect the analyte. Preferably, the matrix layer facilitates the exposure of the indicator molecules to the analyte, and the optical characteristics of the indicator molecules (e.g., the level of fluorescence of fluorescent indicator molecules) are a function of the concentration of the specific analyte to which the indicator molecules are exposed. Alternatively, the matrix layer may be composed of several specialized sublayers, wherein the sublayers have different physical characteristics (e.g., pore size) which may promote or retard tissue ingrowth, or which may pass or block molecules (i.e. analytes) of certain sizes.

Fluorescent molecules may be used in diagnostics as tags and probes when linked to antibodies or other molecules, and can be configured at a molecular level to be used as chemical and biochemical active indicators specifically designed to detect certain analytes, for example glucose. Fluorescent sensors using an anthrylboronic acid-containing compound can be used as a fluorescent chemosensor for signaling carbohydrate binding, including binding of glucose and fructose. Fluorescent molecules are susceptible to degradation, where they lose fluorescence intensity (or brightness) over time by often variable rates of oxidation. The oxidation may be commonly associated with photobleaching, (i.e. photo-oxidation), or may be oxidized by various reactive oxygen species within the local environment of the fluorescent molecule. Inside a living body, normal reactive oxygen species (ROS) are potential oxidants and can include those involved in typical healthy healing reactions such as peroxide, hydroxyl radicals, peroxynitrite, superoxide, and others. Inside a living system there are also specific enzymes called oxygenases for the specific purpose of oxidation in the breakdown of molecules. An adverse result of reactive oxygen species or oxygenase activity on a fluorescent molecule is typically loss of fluorescence. In the case of an indicator molecule, or a passive tag, probe, or label, the useful life and sensitivity of the device, or diagnostic, is limited, or may be rendered completely ineffective by oxidative degradation of fluorescent signal.

Preferred indicator molecules used in embodiments of the invention include those described in U.S. patent application Ser. No. 11/487,435 (U.S. Pat. Pub. No. 2007/0014726, the contents of which are incorporated herein by reference) which are designed to be resistant to oxidation damage from reactive oxygen species. However, one of ordinary skill would recognize that many types of indicators may be used, particularly those described in the patents and publications referred to above (para. [0006]). In a preferred embodiment, the indicator comprises a phenylboronic acid residue.

Rendering the indicator molecules to be resistant from oxidation may be achieved by modifying the indicator molecules and/or hydrogel matrix with a catalytic antioxidant including species such as ascorbic acid, tocopherol, uric acid, glutathione, Salen-Manganese complexes, enzymatic systems (e.g., superoxide dismutase, catalase, glutathione peroxidase), and proteins used to sequester metals capable of HO radical production (e.g., transferrin, ferritin, ceruloplasmin, hemopexin, haptoglobulin, and albumin). The catalytic antioxidant in an embodiment of the invention is a superoxide dismutase or catalase mimic, which grants antioxidant activity to a structure without triggering an immune system response to foreign protein matter. The catalytic antioxidants may be incorporated with the indicator molecules by chemical reaction and/or copolymerization. Copolymerization allows more control over the ratio between antioxidant moieties and indicator moieties and a higher concentration of antioxidant moieties in the indicator macromolecule than attaching antioxidants to the indicator macromolecule by chemical reaction of antioxidant molecules.

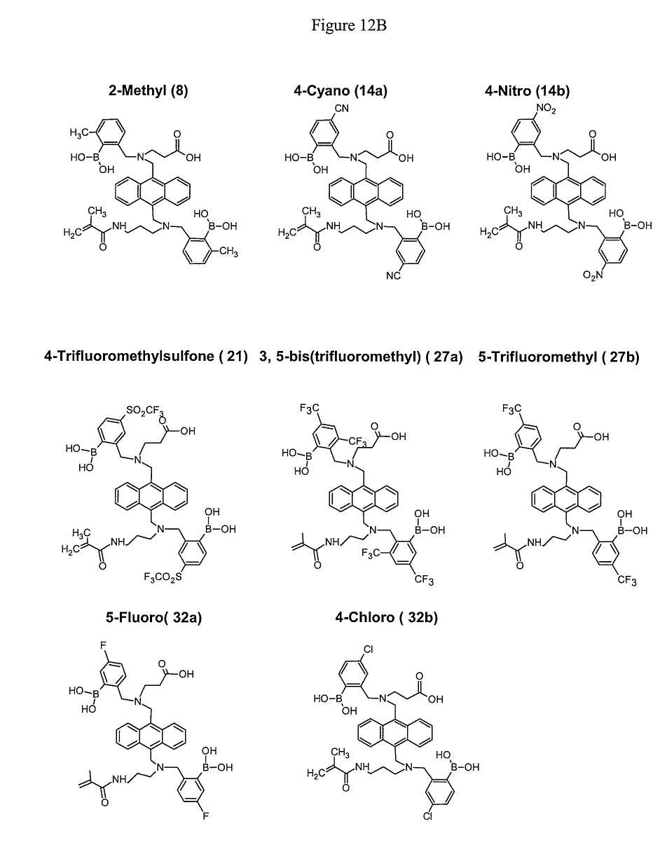

Preferred indicator molecules used in embodiments of the invention may also include those described in U.S. patent application Ser. No. 11/948,419 (U.S. Pat. Pub. No. 2008/0145944) which are designed to include an electron withdrawing group in order to reduce the susceptibility to oxidation of the indicator molecules. In embodiments of the invention, indicator molecules containing an aryl boronic acid residue may be made more resistant to oxidation by adding one or more electron-withdrawing groups to the aromatic moiety which contains the boronic acid residue, thus stabilizing the boronate moiety. It will be understood that the term “aryl” encompasses a wide range of aromatic groups, such as phenyl, polynuclear aromatics, heteroaromatics, polynuclear heteroaromatics, etc. Non-limiting examples include phenyl, naphthyl, anthryl, pyridyl, etc. A wide range of electron-withdrawing groups is within the scope of the invention, and includes, but is not limited to, halogen, cyano, nitro, halo substituted alkyl, carboxylic acid, ester, sulfonic acid, ketone, aldehyde, sulfonamide, sulfone, sulfonyl, sulfoxide, halo-substituted sulfone, halo-substituted alkoxy, halo-substituted ketone, amide, etc., or combinations thereof. Most preferably, the electron withdrawing group is trifluoromethyl. In embodiments of the invention, the electron withdrawing groups of the indicator molecules occupy the R₁ and/or R₂ positions in either of the specific chemical structures of the indicator molecules shown below:

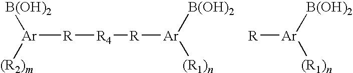

wherein each “Ar” is an aryl group; each R1 and R2 are the same or different and are an electron withdrawing group; “m” and “n” are each independently integers from 1 to 10; R4 is a detectable moiety; and each R is independently a linking group having from zero to ten contiguous or branched carbon and/or heteroatoms, with at least one R further containing a polymerizable monomeric unit. In a particularly preferred embodiment, the indicator comprises one or more of the compounds depicted in FIG. 12. It will also be understood from the above definition that the indicator molecule compounds and detection systems may be in polymeric form.

An implantable device requires a breach of the skin of some size simply to permit insertion of the device. In one embodiment of the present invention, a sensor is implanted through the skin in a procedure to place it within the subcutaneous space between muscle below and dermis above. Necessary mechanical damage occurs to local and adjacent tissue as a result of the foreign body intrusion even for the most biocompatible devices simply because one must first penetrate the skin, and then must displace tissue to create a pocket or space where the device will be deposited and remain in place to execute its intended in vivo function. The relative biocompatibility of the sensor itself, other than its relative size and displacement, does not influence the minimal damage that must be imposed on localized tissue in order to put the sensor or device into place. As a result of foreign body intrusion and localized tissue damage, an immediate and normal inflammation cascade commences within the host in direct response to the intrusion and has the purpose of protecting the host and immediately beginning a repair process to correct the mechanical damage of intrusion, i.e. the wound begins to heal up.

It is observed that when a sensor is placed into an animal, and even more acutely within a diabetic human, there is a near immediate biological response, and a damage inflicted on the extended performance of the sensor by the body as a direct result of inflammation. The net result of the damage from inflammation reaction is to shorten the useful life of the device, for example by diminishing signal strength. For other devices, the reduction in useful life could be measured in terms of response fouling, reducing mechanical strength, electrical or mechanical insulation properties, or according to other measurable properties.

Definitions.

As used herein, “resorb” and “resorbable” and grammatical equivalents of these terms are defined to refer to the process or quality of a material to dissolve and assimilate into a surrounding in vivo environment.

As used herein, “persist” and “persistence” and grammatical equivalents of these terms are defined to refer to the duration of time that a material in an in vivo environment will remain substantively coherent before being broken down and dissolved by the surrounding environment.

As used herein, “explant” and grammatical equivalents of this term are defined to refer to a foreign object (i.e. not biological tissue) which has been implanted into a living body and subsequently removed from that body. An explant may possess biological material that remained attached to the explant after extraction from the living body.

As used herein, “ISF” stands for interstitial fluid.

As used herein, “ROS” stands for radical oxygen species or highly reactive oxygen species.

The Effect of Inflammation Response on an Implanted Device.

Inflammation response is a transient condition occurring in direct response to an injury. There is necessarily a minor tissue injury as a result of implanting a device and it has been observed that the inflammation cascade response can negatively affect an implanted device. Therefore, a solution approach can be to protect the sensor from the moment of implant throughout the transient period of healing. Subsequently, once the inflammation condition surrounding the sensor has subsided, the solution approach can de-protect the sensor to allow it to operate in free equilibrium with its surroundings. In other words, the sensor must be protected from its in vivo microenvironment surroundings during the heal-up period when radical oxygen species and proteins are present, and de-protected after these damage-causing species have subsided during heal-up.

An oversimplified description of the inflammation cascade response to an injury and related foreign material, which in embodiments of the present invention is an implantable device or sensor, is described below. Within seconds following injury, proteins become present in the interstitial fluid (ISF) where they are not otherwise normal solutes. Proteins occur only transiently in ISF as a response or byproduct to injury. These initial proteins, arrive in blood coincident with a cut or any micro-vessel rupture that may cause some blood leakage into the space. Within minutes, swelling begins due to microvasculature dilation. As the pores enlarge due to capillary dilation to allow monocytes and neutrophils into the damage area, serum leaks into the space bringing serum proteins such as albumin and other such proteins. Within hours, generally under chemo-tactic tracking, neutrophils arrive within the space and begin producing reactive oxygen species to break down foreign matter and damaged tissue to initiate repair. After about 1-2 days proteins are no longer a specific threat to the device; they are swept from the ISF and the medium returns to normal. Within about 3-4 days, if the foreign material cannot be destroyed or ejected, a fibrotic capsule of connective tissue is formed around the material (i.e., a sensor or device) to completely encapsulate it, generally completing the inflammation cascade response. When the capsule has formed around the foreign material, there is no further stimulus for reactive oxygen species to attack the capsule itself. Thus, the period of protection that is required is for an interval ranging between the moment of implant through about 4-5 days.

Both protein and ROS are soluble in ISF and diffusible species that attack the surfaces of materials and devices by diffusing into, and either reacting or binding to the surface. A solution to this problem is an applied coating that seals the device against ROS and protein, and dissolves or erodes after a suitable period of time (for example, about 4-5 days) when heal-up inflammation response has substantially subsided, and the damaging species have passed from the microenvironment surrounding the sensor. At the moment of implant, the coating provides a mechanical barrier from either protein or ROS entering the graft matrix where it can either infill the matrix, or attack the indicator system via oxidation. When the temporary protective surrounding coating dissolves away from the sensor after a suitable period of time, the intended analyte sensitivity and function of the sensor is enabled by free equilibrium with fluids and small molecules within the space. The thickness of the coating can vary widely, depending on the identity of the coating and the length of time of the desired protection. The identity of the coating may also vary widely, and preferably includes physiologically compatible materials, such as polymers, that dissolve or degrade over time in in vivo conditions. Such materials may include materials used to make, for example, resorbable sutures. Such materials could also include materials made from processed collagen; poly(glycolic acid), poly(lactic acid), and copolymers thereof; polyanhydrides, etc. The coating may be applied to the sensor material in any suitable fashion, such as by spraying, dipping, thermal extrusion and other such methods of applying or depositing a thin coating layer onto the implant.

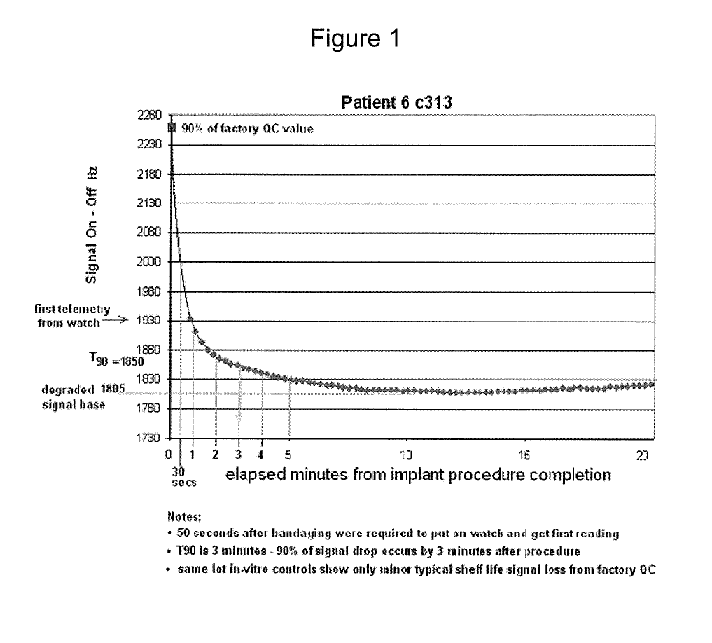

FIG. 1 is a graph shown as an example of near immediate signal loss as a result of biological response to device implantation, where the signal is from an implanted glucose sensor. The data in FIG. 1 were obtained from a sensor that was implanted into a human within the subcutaneous space in the wrist area. This sensor was not treated with a protective coating according to an embodiment of the invention. Within 50 seconds following the completion of the procedure, an external watch reader was placed over the sensor to allow data communication between the sensor and external reader. Data was taken from the sensor at 15 second intervals. It can be seen from FIG. 1 that a very rapid signal drop occurs immediately upon implant where within approximately 3 minutes following the implant procedure (the procedure itself requires approximately 5 minutes) the signal has dropped by 90% of an approximately 20% signal drop overall. This signal drop is undesirable because it shortens the overall useful life of the implant.

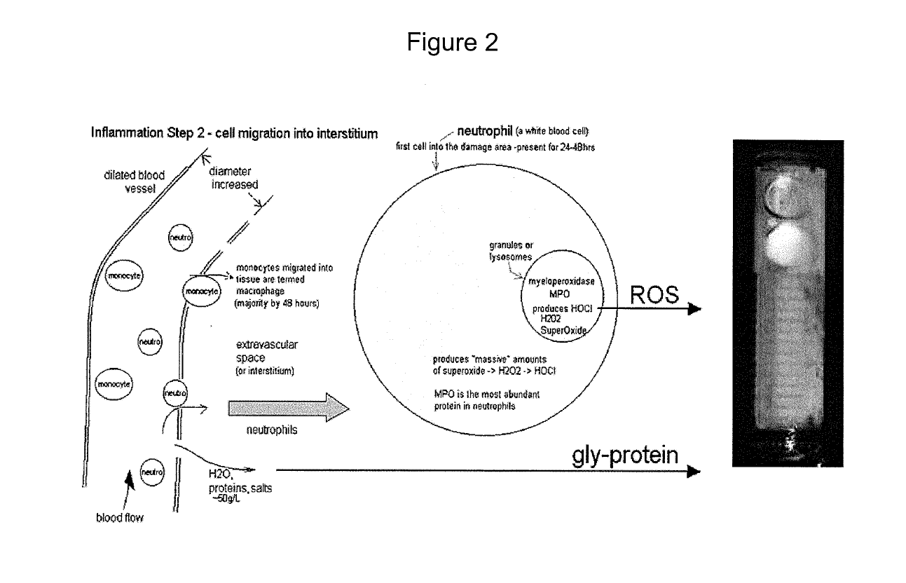

The inflammation reaction is a normal transient cascade response to damage and foreign bodies. As a result of injuring tissue, either by a cut, mashing a finger with a hammer, surgical procedure such as device implantation, or other similar events, the body responds along a known sequence of physiological and biochemical actions. For illustration purposes, the relevant portion of the typical inflammation cascade is shown in FIG. 2.

FIG. 2 is a schematic representation of a living body's inflammation reaction in response to tissue injury. As can be seen from FIG. 2, upon injury, blood vessels within the injury region begin to dilate and increase their diameter. Also, as dilation occurs, pores in the blood vessel walls increase in diameter. These small pores permit fluids, small molecules, and salts to pass through, but are normally too small to permit proteins or cells to pass through from the blood stream into the ISF outside of the blood vessel wall. In response to injury under normal healing, these pores enlarge significantly with blood vessel dilation to permit large monocytes and neutrophils to pass from the bloodstream into the interstitial space in order to protect the body from infection and to initiate repair of the tissue injury. As these pores enlarge to allow cells to pass through, the enlarged pores also allow relatively large volumes of fluids (water) and proteins to pass through into the interstitial space. This increase in fluids moving into the injury site as a result of blood vessel leakage through the expanded pores is commonly observed as swelling to an injury site.

Proteins are not normal solutes within ISF as they are contained within the blood vessel walls. Neutrophils are also not normally within ISF. Both proteins and cells are permitted into ISF as a result of injury. Also, once proteins are leaked into ISF as a result of injury, within a relatively short period of time (less than 24 hours typically) they are swept from the ISF into the lymph system and the ISF is returned to normal (i.e., ISF with no protein present). Neutrophils and any other cells present are also within the interstitial space for a limited amount of time in response to the injury to conduct their particular repair functions. Neutrophils release highly reactive oxygen (radical) species (ROS) which serve to oxidize and break down any damaged tissue and any foreign material to permit the regeneration/repair to complete. These reactive oxygen species also damage the implanted device or sensor by attacking key functional components such as materials and/or chemical indicators.

Embodiments of the present invention address the two major mechanisms by which inflammation reaction can damage a sensor implant placed within the interstitial space. The first mechanism is oxidation by ROS, and the second is protein buildup. The image in the schematic of FIG. 2 shows the effects of ROS oxidation and protein buildup on an embodiment of the invention. Both of these mechanisms are independent, but coincident in occurring as a direct result of inflammation. The present invention considers the timing associated with normal heal-up inflammation cascade, and protects such devices within the time period until inflammation resolves or subsides during the course of injury or wound heal-up surrounding the implant.

Two Mechanisms that Damage Implants: Oxidation by ROS & Protein Infilling from Capillary Leakage.

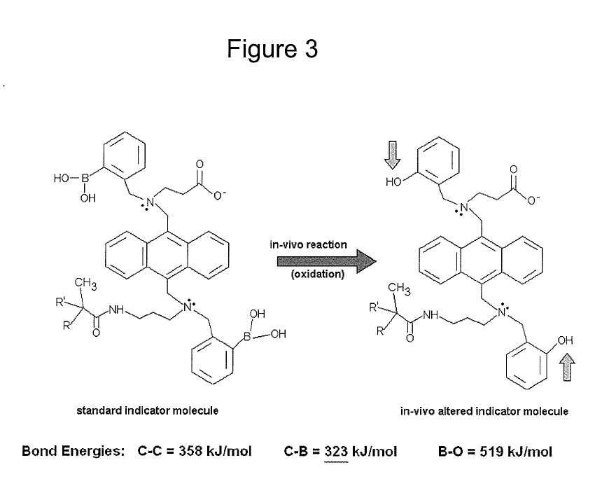

The first mechanism addressed by embodiments of the invention is the loss of signal by indicator oxidation, where the oxidation is caused by ROS. Analysis of sensors explanted from humans (and animals) shows specific and definitive evidence of reactive oxygen species attack. These are the oxygen radicals associated with wound healing including peroxide, superoxide, hypochlorite, peroxynitrite, and hydroxy radical as produced from local repair cells migrated to the site in response to injury. The specific oxidation reaction damage from ROS inflicted on the glucose sensor indicator is shown in FIG. 3.

FIG. 3 represents the in vivo ROS oxidative deboronation reaction of one glucose indicator molecule within the present invention, and shows that as a direct result of ROS produced by repair cell mechanism, the boronate recognition element of the indicator system is converted to a hydroxyl group. The reaction illustrated in FIG. 3 shows the conversion of the standard indicator molecule to the in vivo altered indicator molecule, where the boronate recognition element of the indicator system had been oxidized to a hydroxyl group, thereby causing total loss of activity (specifically, fluorescence modulation) in the molecule. The critical bond energies in the reaction as shown in FIG. 3 are: C-C=358 kJ/mol; C-B=323 kJ/mol; and B-O=519 kJ/mol. These bond energies indicate that the carbon-boron bond, having the lowest bond energy, is most readily susceptible to attack and cleavage by oxidation. This explanted sensor analysis is confirmed by an Alizarin Red assay (negative for boronate), and a Gibbs test (positive for phenol). The loss of boronate from the indicator directly results in loss of fluorescent signal.

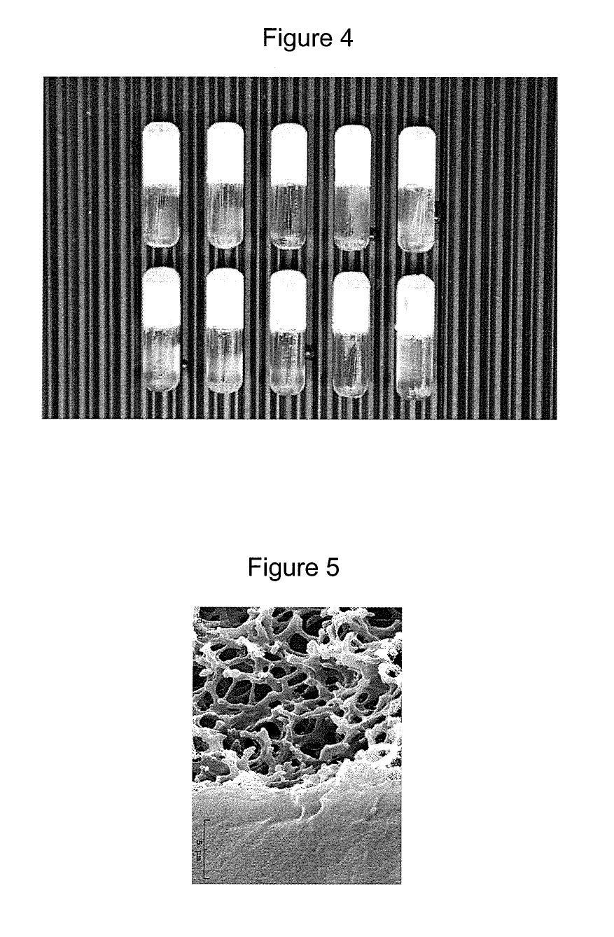

The second mechanism addressed by embodiments of the invention is the loss of signal through loss of optical efficiency caused by in vivo protein infilling. Unwanted protein attachment by either specific or non-specific binding can result in multiple possible compromises in performance of any particular device. In the case of a glucose sensor according to one embodiment of the present invention, unwanted protein binding results in loss of optical efficiency within the sensor's optical system, directly resulting in loss of signal. One sensor according to an embodiment of the invention has a light scattering polymer which provides a 78% increase in signal relative to a clear non scattering polymer formulation. The light from the indicator polymer graft is scattered because the pore texture of the polymer is larger than the wavelength(s) of light that are incident on the graft. This light scattering increases the overall efficiency of the system and gives the graft a white appearance as shown in FIG. 4.

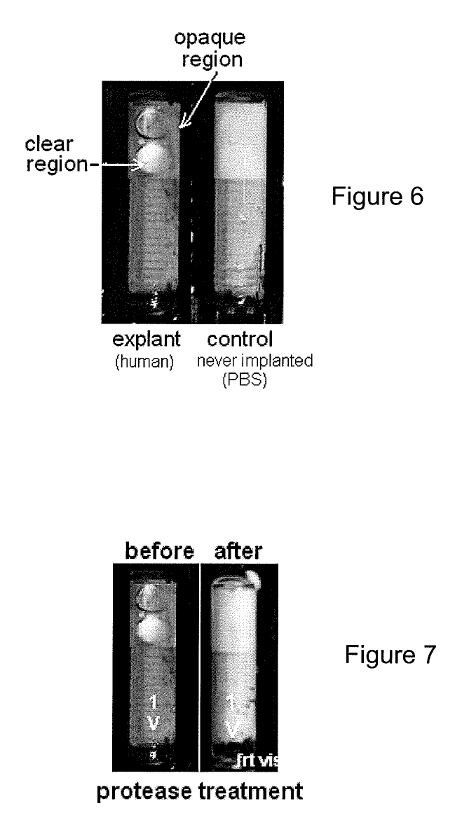

FIG. 4 is a picture of several devices according to embodiments of the invention displaying how light scatters through the sensor indicator graft membrane. The brilliant white regions shown in the Figure are regions on devices according to embodiments of the invention where indicator polymers are grafted onto the clear substrate. The underlying porous structure of the membrane, and the size of the pores (˜1 micron average) create the light scattering effect as shown in FIG. 5.

FIG. 5 is an electron micrograph of the graft membrane structure which is layered on the surface of devices according to the invention. The solid substrate is shown in the bottom-most portion of the photo and the graft in the upper-most portion. If these pores as shown in FIG. 5 become in-filled with protein, the pore sizes are effectively reduced with respect to optics, and the brilliant white light scattering grafts shown in FIG. 4 turn to clear. As the graft turns clear, its ability to scatter light goes to zero, the overall system optical efficiency drops dramatically, and the result is an extreme reduction of fluorescent light arriving at the photodiodes, and signal is thus lost as a direct result of protein infilling into the pores.

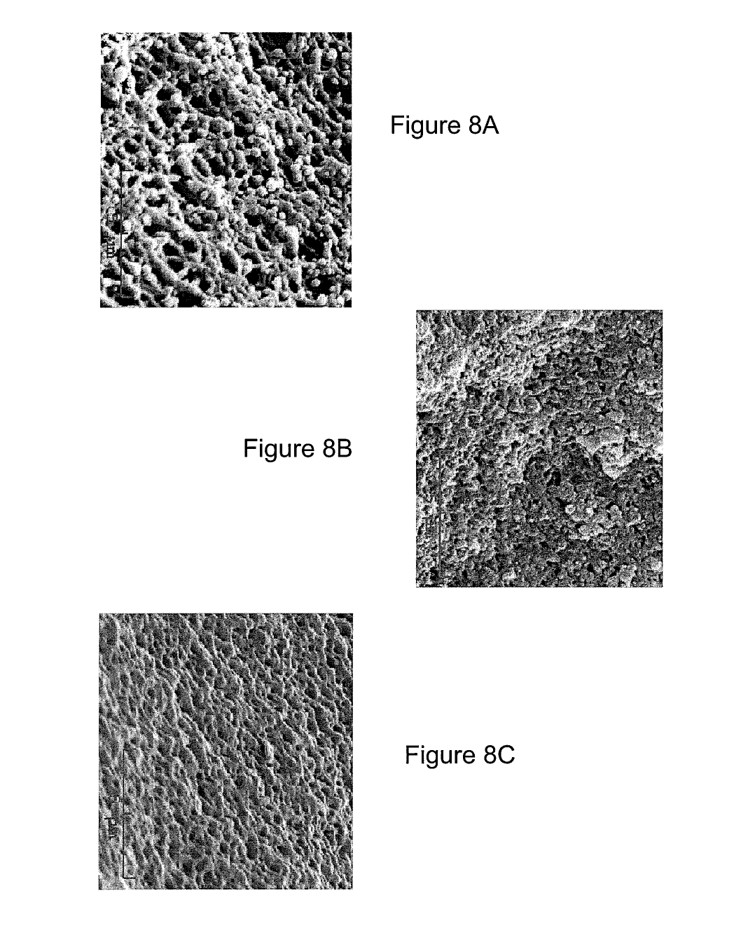

FIG. 6 shows a sensor explanted from a human (on the left) next to a sensor which has never been implanted in a living body (on the right). The explanted sensor on the left side of FIG. 6 shows where there are both clear and opaque regions remaining within the overall area of the indicator membrane graft.

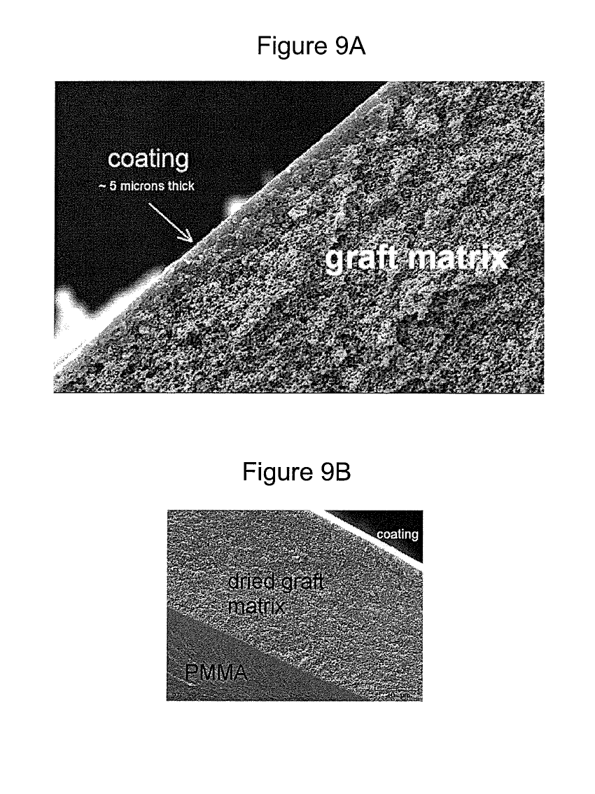

FIG. 7 provides evidence that the clear region is caused by protein infilling is supported by treatment of the sensor with a protease. FIG. 7 shows the same explanted sensor, where the left side of the image shows the sensor after implantation and removal, and the right side of the image shows the sensor following a subsequent treatment with protease. The protease digests any protein within the graft and the result is that the clear region of the sensor reverts to being opaque as shown in the right side image of FIG. 7.

Prevention of ROS Deboronation & Protein Infilling.

Both of above-described mechanisms, ROS oxidation and protein infilling of the graft pores, are the result of normal heal-up inflammation under stimulus of implanting the sensor under the skin and the attendant disruption and small damage to localized tissue. Both result directly in loss of signal, thereby shortening the useful life of a sensor (or other susceptible device/material).

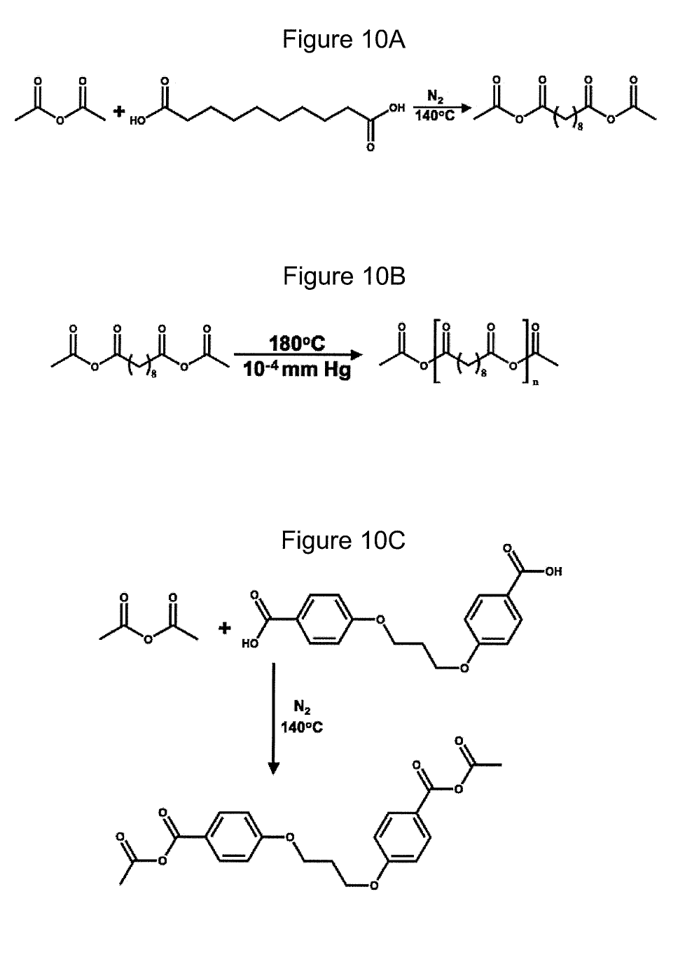

Since optical scatter is mechanical (i.e., based on pore size and texture) and not chemical, it is possible to visualize by electron microscopy the difference between the clear and opaque regions on the human sensor explant as shown in SEMS images of FIGS. 8A-8C. FIGS. 8A, 8B, and 8C are SEMS images of a sensor which has never been implanted (control), the opaque region of a human explant, and the clear region of a human explant, respectively. FIG. 8C shows the surface of an explant with infilling of graft pores, which results in that region being clear.

According to embodiments of the invention, a resorbable coating is applied to implantable devices which will prevent protein infilling of graft membrane structure as well as prevent ROS from deboronating the glucose indicator. The coating is designed to persist for a period of time under in vivo conditions, after which the coating will dissolve or erode and dissociate from an implanted device and resorb into the surrounding environment. The coating is designed to have a persistence such that the coating protects the device for the desired period of time (e.g., about four to about five days) and then separates from the device in its entirety, allowing the device to perform its in vivo functionality unencumbered from residual coating that may inhibit the flow or diffusion of the target analyte to the indicator molecules. In embodiments of the invention, the protective coating may range in thickness from about 5 microns to about 200 microns, more preferably from about 5 to about 80 microns, and in more preferred embodiments the thickness of the protective coating will be in the range of about 20-30 microns.

The coating may be composed of different materials including polyesters and polyanhydrides, which in embodiments of the present invention have molecular weights ranging from about 15 kDa to about 100 kDa. The materials used for the protective coating are soluble in vivo at molecular weights of less than 1,000 Da. The choice of coating material allows for control of how long the coating will take to dissolve and resorb in vivo, as the materials are resorbed via homogeneous and heterogeneous erosion. For example, polyesters are randomly cleaved near their midpoint, thus the polyester strands of a coating will be dissolved in cycles, being cleaved into smaller chains followed by further dissolution until the molecular weight reaches about 1 kDa, which is water soluble. In contrast, polyanhydrides are generally cleaved at the surface, releasing relatively small and soluble molecules at a quicker rate than the cycle that cleaves polyesters. Relatively, aliphatic polyanhydrides dissolve quicker than polyesters, and this difference allows for design of a coating that will persist for a longer or shorter period of time in vivo, based on molecular weight.

According to an embodiment of the invention, the protective coating may be applied to the device by a spraying method. It has been observed that application of a protective coating by spraying allows for excellent control of thickness. In a preferred embodiment of the invention, the protective coating is about 20-30 microns thick, and can be accurately applied by spraying the protective coating. Conversely, for a device that is designed to take in some degree of fluid, such as the matrix layer of indicator molecules in embodiments of the invention, the process of spraying the protective coating may cause the fine liquid droplets of polymer solvent to wick into the matrix layer and block where indicator molecules would otherwise accept target analytes. Thus, in some embodiments it is desirable to control the spraying application process to minimize any capillary action effects between the polymer solvent and susceptible surfaces.

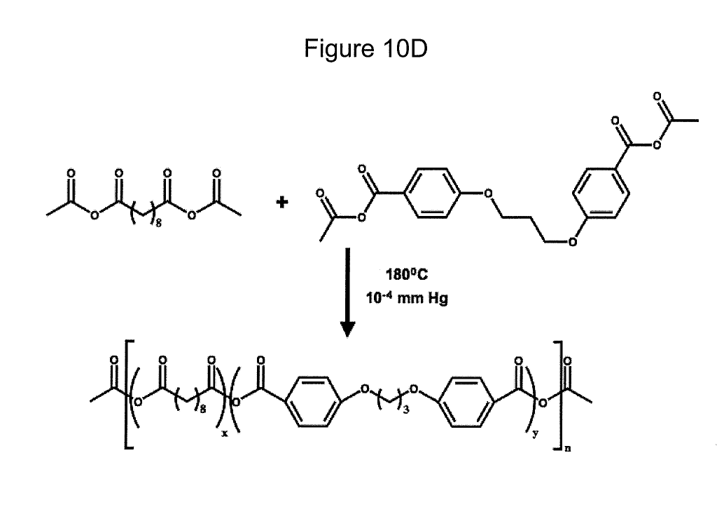

According to an embodiment of the invention, an approximately five micron (5 μm) thick spray coating of poly(lactic-co-glycolic acid) is applied to the surface of the graft and shown in the electron micrographs of FIGS. 9A & 9B. The coating thickness of about five microns has been established empirically at 37° C. as one that will protect the sensor from diffusion of ROS and protein, and then dissolve or erode and expose the graft matrix to the surroundings within about 3-4 days following implant.

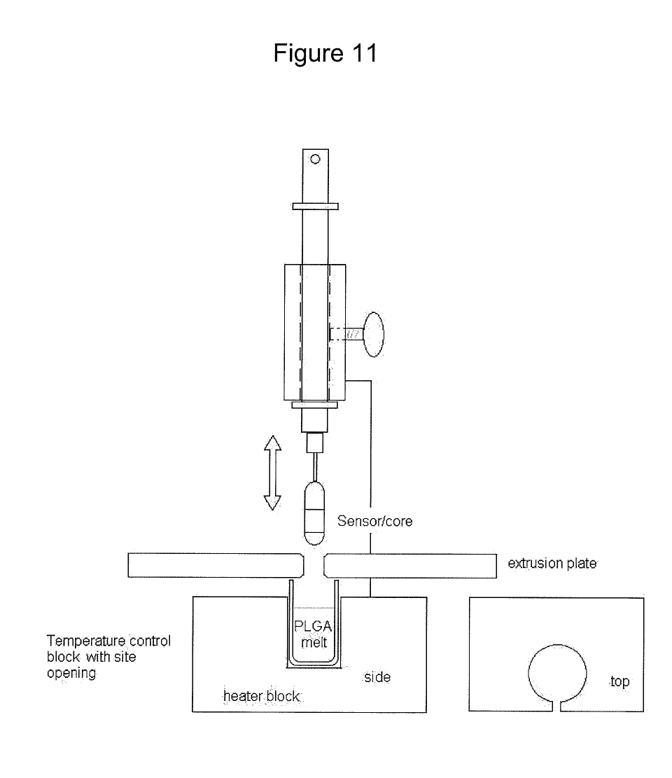

According to an embodiment of the invention, a protective coating comprised of polyanhydrides is applied to the device, wherein the process involves the solvent dip-coating of polyanhydrides to form a water impenetrable barrier on the device. A variety of synthesized aliphatic and aromatic polyanhydride homo- and copolymers can be used as a water impenetrable barrier for the protection of the indicator from the body's inflammatory reactive oxygen species (ROS) response or oxidation. According to embodiments of the invention, these species can include poly(sebacic acid) and poly(1,3-bis(p-carboxyphenoxy)propane-co-sebacic acid) coating as a moisture barrier for protection against oxidation. The synthesis of these species is represented in FIGS. 10A-10D.

In an embodiment of the invention, poly(sebacic acid) (PSA) (1.0 g) was placed in a 20 mL scintillation vial followed by addition of ethyl acetate (5 mL). The solution was heated to 60° C. in order to dissolve the poly(sebacic acid). The warm solution was then utilized to yield a polymer coating on the surface of the sensor. The sensor was dipped into the warm PSA solution with a dwell time of 5 seconds followed by a 5 second removal time. The sample was allowed to dry in ambient conditions for 30 seconds and the dip process aforementioned was repeated two more times. The sensor was then placed in an 80° C. oven for 1 hour in order to anneal the PSA coating and remove the residual ethyl acetate solvent.

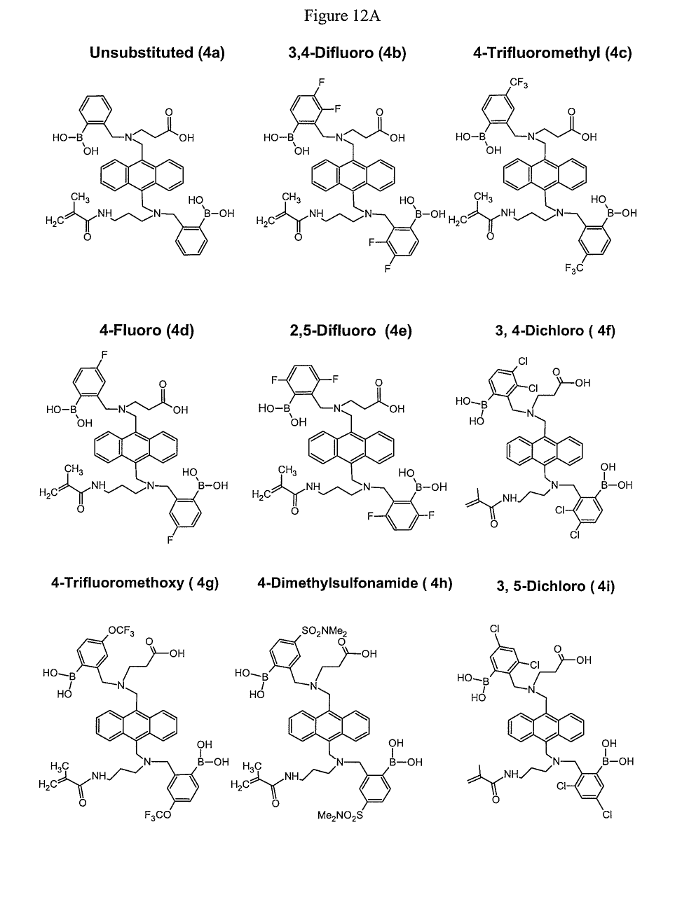

According to another embodiment of the invention, the protective coating may be composed of poly(D,L-lactic-co-glycolic acid) and applied to the device by a thermal extrusion method. An embodiment of the invention applies a uniform coat of resorbable poly(DL-lactic-co-glycolic acid) (“PLG”) onto a device, such as a glucose sensor or device, in order to provide temporary protection from ROS species under in vivo conditions. The protective material may be 50/50 poly(DL-lactic-co-glycolic acid) copolymer, preferably obtained from Purac Biomaterials from Lincolnshire, Ill. (Brand name: PURASORB PDLG 5002). The extrusion procedure itself is conducted on a semi-automated custom built extrusion set up comprising a heating block, extrusion plate and up-down slide jig as represented in the schematic of FIG. 11.

The extrusion procedure takes grafted and lathed sensors which are inspected and photographed before applying PLG coating. Initial outer diameter (OD) measurements of undercut area of each sensor are taken. The PLG is melted in a 140° C. oven and degassed while hot by centrifuging prior to use. During the coating procedure the molten PLG and extrusion plate are kept at about 110-120° C. Other polymers may be used in other embodiments of the invention, and those polymers may be kept at different temperatures, along with the extrusion plate, as appropriate to achieve the goals of the method. The sensors are secured to a slide jig and centered above the appropriate extrusion hole. The sensor is dipped slowly into molten PLG until fully submerged. The dwell time of the sensor in the molten PLG is set to be 5 seconds. Then the sensor is withdrawn through the hot extrusion hole such that the excess PLG is scraped away. After extrusion the sample is held above the plate for about 2 minutes to allow the PLG to cool down and harden. After the initial 2 minute cooling period, a second PLG coat is applied. Final OD measurements of undercut area of dried sensors/cores are taken and the thicknesses of PLG coating are calculated. The coated sensors are stored overnight to ensure curing of PLG before they are sent for ethylene oxide (ETO) sterilization.

Accordingly, it should be understood that a variety of applications, modifications, and variations can be made by those in the art within the scope of the following claims.

1. A device comprising:
(a) an implantable device which has an in vivo functionality and;
(b) a layer of protective coating applied onto the implantable device wherein:
(1) the protective coating prevents or reduces degradation or interference of the implantable device from inflammation reactions; and
(2) the protective coating is designed to resorb over a period of time under in vivo conditions.

2. The device of claim 1, wherein the device is a sensor.

3. The device of claim 2, wherein the sensor is for monitoring blood glucose levels.

4. The device of claim 1, wherein the protective coating is applied to the device by dipping the device in the coating material.

5. The device of claim 1, wherein the protective coating is applied to the device by spraying the coating onto the device.

6. The device of claim 1, wherein the protective coating is applied to the device by thermal extrusion of the coating onto the device.

7. The device of claim 1, wherein the protective coating is a physiologically compatible material comprising one or more polyanhydrides.

8. The device of claim 7, wherein the one or more polyanhydrides comprises poly(sebacic acid) and/or poly(1,3-bis(p-carboxyphenoxy)propane-co-sebacic acid).

9. The device of claim 1, wherein the protective coating is a physiologically compatible material comprising processed collagen.

10. The device of claim 1, wherein the protective coating is a physiologically compatible material comprising one or more polyesters.

11. The device of claim 10, wherein the one or more polyesters comprises polyglycolic acid and/or polylactic acid, or copolymers thereof.

12. The device of claim 1, wherein the layer of protective coating is from about 5 to about 200 microns thick.

13. The device of claim 12, wherein the layer of the protective coating is from about 20 to about 30 microns thick.

14. The device of claim 1, wherein the period of time is from about 4 to about 5 days.

15. The device of claim 2, wherein the sensor comprises a body surrounding a photosensitive detector element and a light source, and further wherein the external surface of the sensor body comprises a matrix layer that comprises one or more indicator molecules.

16. The device of claim 15, wherein the one or more indicator molecules comprises a phenylboronic acid residue.

17. The device of claim 15, wherein the sensor further comprises a power source and a transmitter surrounded by the sensor body.

18. A method for using an implantable device in in vivo applications comprising:
(a) providing an implantable device which has an in vivo functionality, and which comprises a layer of a protective coating on the device wherein:
(1) the protective coating prevents or reduces degradation or interference of the device from inflammation reactions; and
(2) during use the protective coating resorbs into the surrounding environment over a period of time; and
(b) implanting the implantable device in a subject body.

19. The method of claim 18, wherein the in vivo functionality of the implantable device is to operate as a sensor designed to detect a target analyte.

20. The method of claim 19, wherein the in vivo functionality of the implantable device is to operate as a sensor designed to detect glucose.

21. The method of claim 18, wherein the protective coating is applied by spraying the protective coating onto the implantable device.

22. The method of claim 18, wherein the protective coating is applied by dipping the implantable device into a reservoir of the protective coating.

23. The method of claim 18, wherein the protective coating is applied by thermal extrusion onto the implantable device.

24. The method of claim 18, wherein the protective coating comprises one or more of processed collagen, polyesters, or polyanhydrides.

25. The method of claim 18, wherein the protective coating is from about 5 to about 200 microns thick.

26. The method of claim 25, wherein the protective coating is from about 20 to about 30 microns thick.

27. The method of claim 18, wherein the protective coating resorbs into the surrounding environment over a period of about 4 to about 5 days.

28. The method of claim 19, wherein the sensor comprises a sensor body surrounding a photosensitive detector element and a light source, and further wherein the external surface of the sensor body comprises a matrix layer that comprises one or more indicator molecules.

29. The method of claim 28, wherein the one or more indicator molecules comprises a phenylboronic acid residue.

30. The method of claim 28, wherein the sensor further comprises a power source and a transmitter surrounded by the sensor body.

31. A method for detecting the presence or concentration of an analyte in an in vivo sample, said method comprising:
a) exposing the sample to a device having a detectable quality that changes when the device is exposed to the analyte, said device comprising a layer of protective coating applied onto the implantable device wherein:
(1) the protective coating prevents or reduces degradation or interference of the device from inflammation reactions; and
(2) the protective coating is designed to resorb over a period of time under in vivo conditions,
such that the device has enhanced resistance to degradation or interference as compared to a corresponding device without the protective coating; and
b) measuring any change in said detectable quality to thereby determine the presence or concentration of said analyte in said sample.

32. The method of claim 31, wherein the analyte is glucose.

33. The method of claim 31, wherein the protective coating comprises one or more of processed collagen, polyesters, or polyanhydrides.

34. The method of claim 31, wherein the protective coating is from about 5 to about 200 microns thick.

35. The method of claim 34, wherein the protective coating is from about 20 to about 30 microns thick.

36. The method of claim 31, wherein the protective coating resorbs into the surrounding environment over a period of about 4 to about 5 days.

37. The method of claim 31, wherein the device comprises a sensor body surrounding a photosensitive detector element and a light source, and further wherein the external surface of the sensor body comprises a matrix layer that comprises one or more indicator molecules.

38. The method of claim 37, wherein the one or more indicator molecules comprises a phenylboronic acid residue.

39. The method of claim 37, wherein the sensor further comprises a power source and a transmitter surrounded by the sensor body.
